(19) United States
(12) Patent Application Publication
(10) Pub. No.: US 2010/0310870 A1
(43) Pub. Date: Dec. 9, 2010

(54) AMORPHOUS BAZEDOXIFENE ACETATE AND PREPARATION THEREOF

(75) Inventors: Shankar Sanganabhatla, Navi Mumbai (IN); Sachin Srivastava, Navimumbai (IN); Dinesh Bansilal Deore, NaviMumbai (IN); Anthony Melvin Crasto, Thane (IN); Mubeen Ahmed Khan, Navi Mumbai (IN)

Correspondence Address: GLENMARK GENERICS INC., 750 CORPORATE DRIVE, MAHWAH, NJ, 07430, US

(73) Assignee: GLENMARK GENERICS LTD., Mumbai (IN)

(21) Appl. No.: 12/724,432

(22) Filed: Mar. 16, 2010

Related U.S. Application Data
(60) Provisional application No. 61/230,800, filed on Aug. 3, 2009.

(30) Foreign Application Priority Data
Jun. 3, 2009 (IN) 1346/MUM/2009

(51) Int. Cl. B32B 5/00; C07D 403/12

(52) U.S. Cl. 428/402; 540/602

(57) ABSTRACT

The present invention relates to amorphous form of bazedoxifene acetate and a process for the preparation thereof.

### PRIORITY

This application claims the benefit under 35 U.S.C. §119 to Indian Provisional Application 1346/MUM/2009, filed on Jun. 3, 2009, and to U.S. Provisional Application 61/230,800, filed Aug. 3, 2009, the contents of which are incorporated by reference herein.

### BACKGROUND OF THE INVENTION

1. Technical Field

The present invention relates to amorphous form of bazedoxifene acetate and a process for the preparation thereof.

2. Description of the Related Art

Bazedoxifene acetate is a third generation selective estrogen receptor modulator and was approved in Europe in 2009 for the treatment of postmenopausal osteoporosis in women at increased risk of fracture. Bazedoxifene acetate, (1-[4-(2-azepan-1-yl-ethoxy)-benzyl]-2-(4-hydroxy-phenyl)-3-methyl-1H-indol-5-ol acetic acid), is structurally represented as

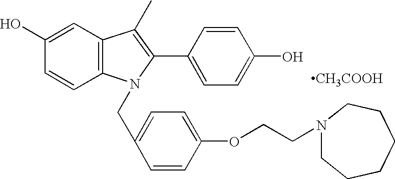

U.S. Pat. No. 5,998,402 describes 2-phenylindoles, including bazedoxifene and their pharmaceutically acceptable salts, a pharmaceutical composition and method of treatment.

U.S. Publication No. 2005227965 discloses form A of bazedoxifene acetate and processes for the preparation thereof.

U.S. Publication No. 2005250762 discloses from B of bazedoxifene acetate and processes for the preparation thereof.

Amorphous forms of active pharmaceutical ingredients (APIs) provide opportunities to improve the performance characteristics, inclusive of solubility, stability, flowability, tractability and compressibility of drug substances, and the safety and efficacy of drug products of a pharmaceutical product. Such discoveries enlarge the repertoire of materials that a formulation scientist has available for designing, for example, a pharmaceutical dosage form of a drug with a targeted release profile or other desired characteristic.

Generally, amorphous solids offer opportunities for solubility and bioavailability enhancement since these materials are more soluble than the crystalline form of the same compound. The rate of dissolution is also a consideration in formulating syrups, elixirs and other liquid medicaments.

The process described herein, for the preparation of amorphous form of bazedoxifene acetate is commercially scalable, simple, convenient and robust.

### SUMMARY OF THE INVENTION

The present invention relates to amorphous form of bazedoxifene acetate and processes for the preparation thereof.

In one aspect, the present invention provides amorphous bazedoxifene acetate having less than about 5% of crystalline forms of bazedoxifene acetate.

In another aspect, the present invention provides a substantially pure amorphous form of bazedoxifene acetate having less than about 5%, of any of crystalline forms A and B of bazedoxifene acetate.

In yet another aspect, the present invention provides amorphous bazedoxifene acetate, having an X-ray powder diffraction (XRPD) pattern, which is substantially in accordance with FIG. 1; and a Differential Scanning Calorimetry (DSC) thermogram, which is substantially in accordance with FIG. 2.

In still yet another aspect, the present invention provides amorphous bazedoxifene acetate further characterized by Infrared (IR) spectrum, which is substantially in accordance with FIG. 3 and Thermogravimetric Analysis (TGA) scan, which is substantially in accordance with FIG. 4.

In a further aspect, the present invention provides a process for the preparation of amorphous bazedoxifene acetate comprising:

a) providing a solution of bazedoxifene acetate in a solvent or mixture of solvents or aqueous mixtures thereof; and

b) removing the solvents to obtain the substantially pure amorphous bazedoxifene acetate.

In another aspect, the present invention provides amorphous particles of bazedoxifene acetate having a specific surface area from at least about 1 m²/g to at least about 3 m²/g, as measured by BET (Brunauer-Emmett-Teller) method.

In one aspect, the present invention provides 90% of amorphous particles of bazedoxifene acetate having particle size less than about 260 μm.

In another aspect, the present invention provides 50% amorphous particles of bazedoxifene acetate having particle size less than 75 μm.

In another aspect, the present invention provides 10% amorphous particles of bazedoxifene acetate having particle size less than 25 μm.

In another aspect, the present invention provides amorphous form of bazedoxifene acetate having plate like crystal shape as observed by Scanning electron micrograph (SEM), substantially in accordance with FIG. 5.

In another aspect, the present invention provides a pharmaceutical composition comprising a therapeutically effective amount of amorphous bazedoxifene acetate, prepared in the process herein described, and at least one pharmaceutically acceptable carrier.

### BRIEF DESCRIPTION OF THE DRAWINGS

FIG. 1: X-ray powder diffraction (XRPD) pattern of bazedoxifene acetate.

FIG. 2: Differential scanning calorimetry (DSC) endotherm of bazedoxifene acetate.

FIG. 3: Infrared (IR) spectrum of bazedoxifene acetate.

FIG. 4: Thermogravimetric analysis (TGA) scan of bazedoxifene acetate.

FIG. 5: Scanning electron micrograph (SEM) of bazedoxifene acetate.

### DETAILED DESCRIPTION OF THE INVENTION

The present invention relates to amorphous form of bazedoxifene acetate and a process for the preparation thereof.

The degree of crystallinity of a crystalline material is established using powder X-ray diffraction. The integrated peak intensity of the crystalline peaks divided by the overall integrated area of the pattern is used to deduce the percent of the crystalline portion. Crystalline peaks produced by an X-ray diffraction measurement, are characterized by having a half-value width below 2 degrees.

Amorphous solids, in contrast to crystalline forms, do not possess a distinguishable crystal lattice. Amorphous solids do not have an orderly arrangement of structural units that correlate with a definitive X-ray diffraction pattern (XRD). Further, amorphous materials fail to give rise to a melting point and tend to liquefy at some point beyond the glass transition point, as would crystalline materials tested with DSC. Advantageously, however, amorphous forms of compounds are generally more soluble than their crystalline counterparts, with a subsequent increased bioavailability thus lending a desirable usage for pharmaceutical formulations.

The present invention provides amorphous bazedoxifene acetate having less than about 20% of crystalline bazedoxifene acetate by weight, more preferably less than about 10% by weight and even more preferably less than about 5% by weight.

The present invention provides amorphous bazedoxifene acetate having less than about 5% of crystalline forms of bazedoxifene acetate.

The present invention provides a substantially pure amorphous form of bazedoxifene acetate having less than about 5%, of any of crystalline forms A and B of bazedoxifene acetate.

The phrase, “substantially pure”, as used herein, is intended to mean an amorphous form of bazedoxifene acetate containing less than 5% of any of the crystalline forms of bazedoxifene acetate. Further, the XRD scan, which shows that the substantially pure amorphous form of bazedoxifene acetate, is having less than about 5%, of any of crystalline forms; and/or is free of XRD peaks from any crystalline form.

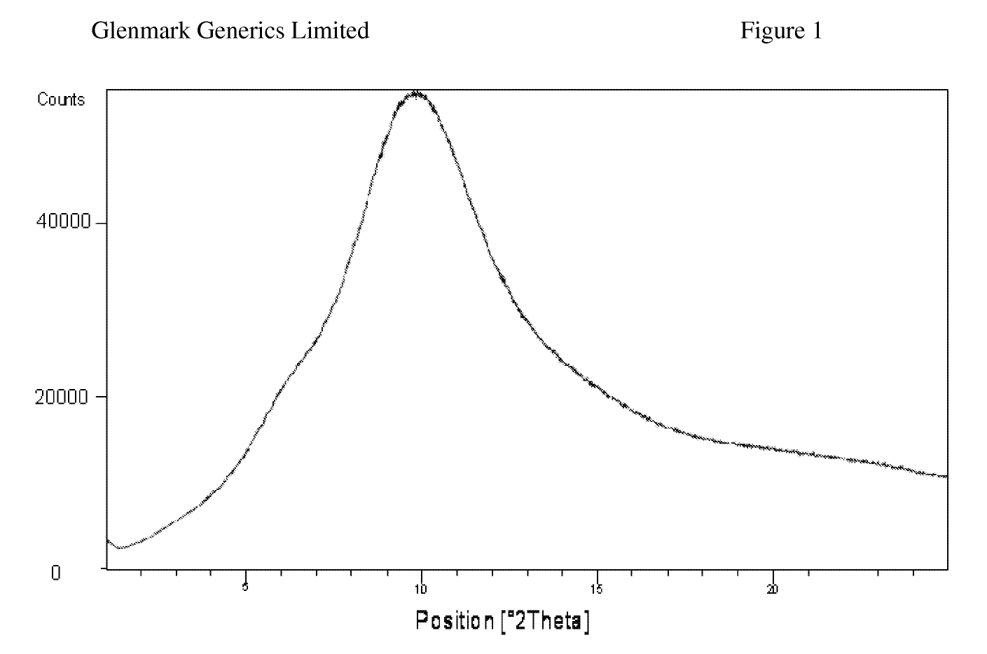

The present invention provides amorphous bazedoxifene acetate having an X-ray diffraction pattern, substantially in accordance with FIG. 1.

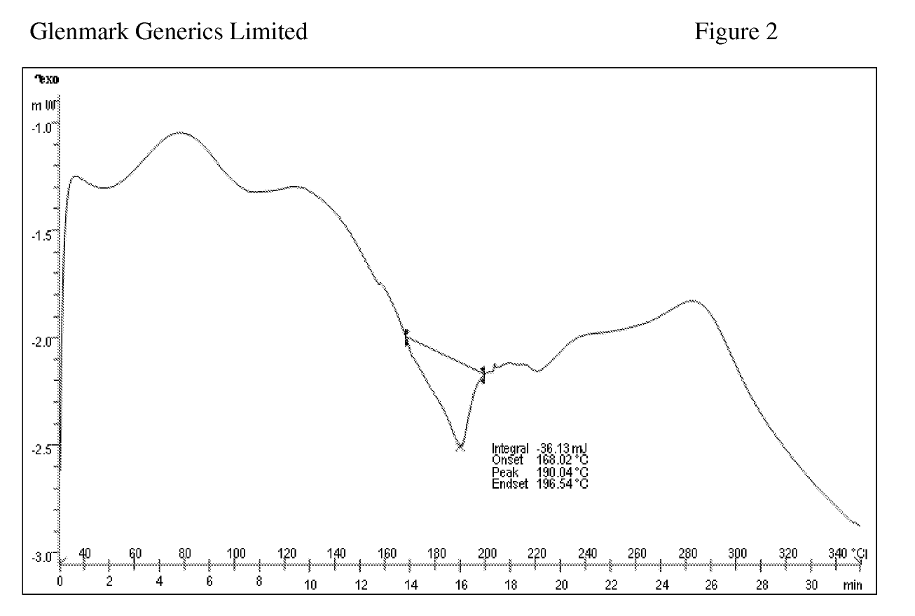

In one aspect, the present invention provides an amorphous form of bazedoxifene acetate having a Differential scanning calorimetry (DSC) thermogram with an endothermic curve at about 190.04° C. with an onset at about 168.02° C. and endset at about 196.54° C., which is substantially in accordance with FIG. 2.

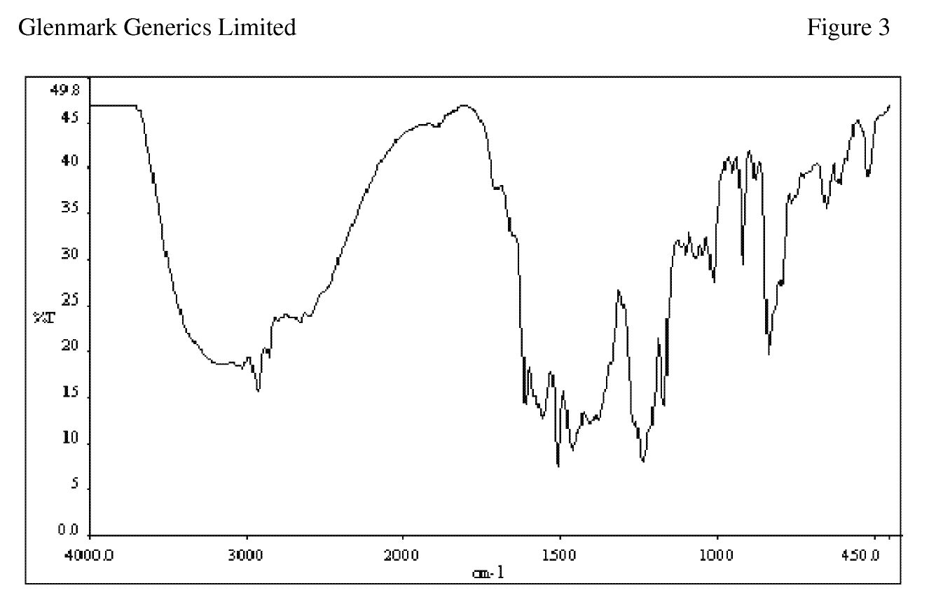

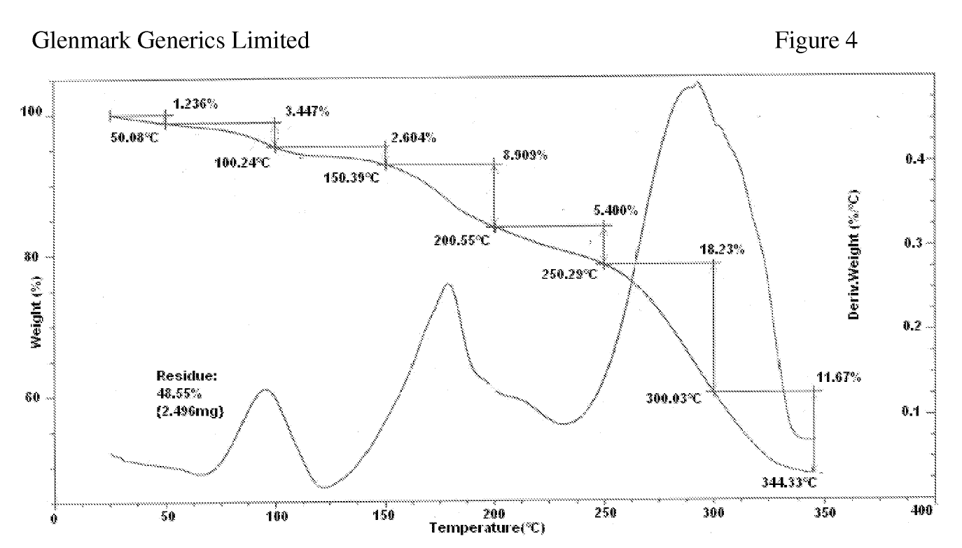

In one aspect, the present invention provides an amorphous form of bazedoxifene acetate having an Infrared (IR) spectrum, substantially in accordance with FIG. 3. The present invention provides an amorphous form of bazedoxifene acetate having a Thermogravimetric Analysis (TGA) thermogram showing a weight loss of about 3.45 percent up to a temperature of 100° C., which is substantially in accordance with FIG. 4.

The present invention provides a process for the preparation of amorphous bazedoxifene acetate comprising:

a) providing a solution of bazedoxifene acetate in a solvent or mixture of solvents or aqueous mixtures thereof; and

b) removing the solvents to obtain the substantially pure amorphous bazedoxifene acetate

The solution of bazedoxifene acetate may be prepared in at least one organic solvent and/or mixture with water; optionally, filtering the solvent solution to remove any extraneous matter and removing the solvent from the resulting solution to obtain amorphous bazedoxifene acetate.

Suitable organic solvents include, but are not limited to, alcoholic solvents having from 1 to 6 carbon atoms such as ethyl alcohol, isopropyl alcohol; water, non-aromatic solvents such as chlorinated solvents, ketones, esters such as isopropyl acetate, protic solvents like DMF (dimethyl formamide), DMSO (dimethyl sulfoxide), DMAC (dimethylacetamide) the like; and mixture thereof. Preferably, the solvent/s are alcoholic solvents, ester, water and mixtures thereof.

Bazedoxifene acetate can be present in any amount that will produce the amorphous form. Preferably, bazedoxifene acetate is present in an amount of about 1% to about 30% by weight of the solvent, more preferably about 1% to about 20% by weight of the solvent, more preferably about 1% to about 10% by weight of the solvent. One skilled in the art would recognize that with the modifications necessary relative to the choice of a solvent or solvents, the amount of bazedoxifene acetate may, likewise, vary.

The solution may be heated to dissolve bazedoxifene acetate. The temperature suitable for dissolving bazedoxifene acetate depends on the solvent or solvents used and the amount of bazedoxifene acetate in the solution. Typically, the temperature is at least about 30° C. to about reflux. Preferably, the temperature is about 40° C. to about 100° C., and more preferably at about 40° C. to about 80° C. The solution may be prepared at other suitable temperatures as long as bazedoxifene acetate is sufficiently dissolved. Increasing the amount of bazedoxifene acetate would generally require the use of higher temperatures. Routine experimentation will provide the approximate range of suitable temperatures for a given solvent and amount of bazedoxifene acetate. The resulting solution may optionally be filtered to remove any extraneous matter present in the solution using any standard filtration techniques known in the art.

Removal of the solvent from the resulting solution is accomplished by, for example, substantially complete evaporation of the solvent, concentrating the solution, cooling to obtain amorphous form and filtering the solid under inert atmosphere. Evaporation can be achieved at sub-zero temperatures by a lyophilisation or freeze-drying technique. The solution may also be completely evaporated in, for example, a Rotavapor, a vacuum paddle dryer or in a conventional reactor under vacuum above about 720 mm Hg or by flash evaporation techniques by using an agitated thin film dryer (ATFD), or evaporated by spray drying to obtain a dry amorphous powder. Preferably, the solvent removal method is lyophilisation.

The bazedoxifene acetate substantially in amorphous form, obtained by the process herein described, may be further dried in, for example, vacuum tray dryer, rotocon vacuum dryer, vacuum paddle dryer or pilot plant rotavapor, to further lower residual solvents. When drying is implemented, the preferred instrument is a vacuum tray dryer.

The specific surface area of an active pharmaceutical ingredient may be affected by various factors. It is recognized that there is an inverse relationship between surface area and particle size; where the smaller the particle size, the higher the surface area. Whereupon, the available surface area for drug dissolution correlates to the rate of dissolution and solubility. A greater surface area enhances both the solubility and the rate of dissolution of a drug, which in turn, may improve its bioavailability and potentially its toxicity profiles.

Thus, there is a need in the art to prepare active pharmaceutical ingredients, such as bazedoxifene acetate, with a high surface area to obtain formulations with greater bioavailability, and to compensate for any loss of surface area before formulation.

Specific surface area is defined in units of square meters per gram (m²/g). It is usually measured by nitrogen absorption analysis. In this analysis, nitrogen is absorbed on the surface of the substance. The amount of the absorbed nitrogen (as measured during the absorption or the subsequent desorption process) is related to the surface area via a formula known as the BET formula.

The present invention provides amorphous bazedoxifene acetate, prepared by the process previously described, having specific surface area from about 1 m²/g to about 3 m²/g, as measured by BET method.

In the field of pharmaceutical formulation, it is notable that particle size plays a pivotal role in the solubility properties of an API, like bazedoxifene acetate. Particle size reduction techniques are commonly employed to increase a compound's solubility. Particle size reduction increases the surface area of the solid phase that is in contact with the liquid medium. However, particle size reduction cannot alter the solubility of the compound in a solvent, which is a thermodynamic quantity. At instances where the rate of dissolution of a poorly soluble drug is the rate limiting factor in its rate of absorption by the body, it is recognized that the bioavailability of such drugs may be enhanced when administration occurs in a finely divided state. Further, particle size can also affect how freely crystals or a powdered form of a drug will flow past each other, which in turn, has consequences in the production process of pharmaceutical products containing the drug.

In one aspect, the present invention provides amorphous bazedoxifene acetate further characterized by Malvern Mastersizer 2000® that demonstrates that the material comprises irregularly shaped particles with 90% of the particles having a particle size of less than 260 μm, 50% of the particles having a particle size of 75 μm and 10% of the particles having a particle size of 25 μm.

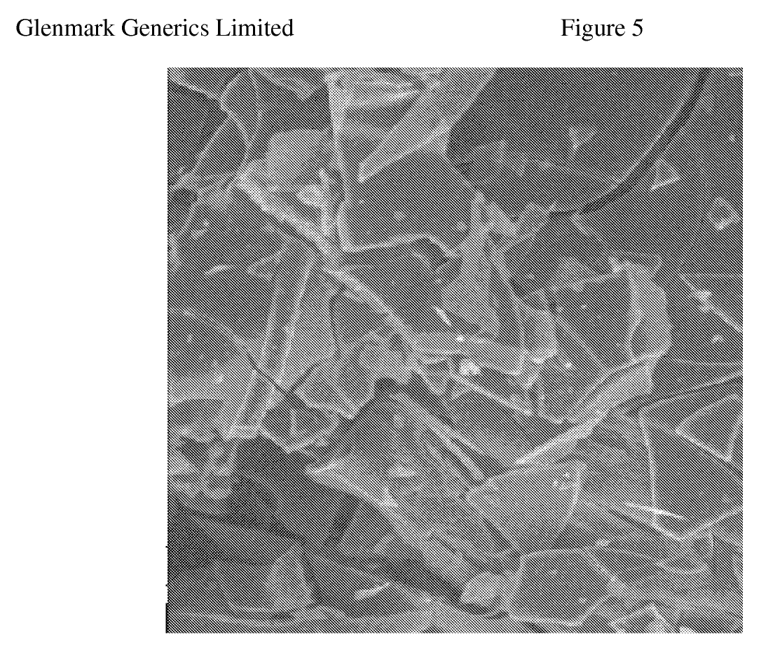

In one aspect, the present invention provides amorphous bazedoxifene acetate having plate-like crystals, as characterized by Scanning Electron Micrograph (SEM), which is substantially in accordance with FIG. 5.

In one aspect, the present invention provides pharmaceutical compositions comprising amorphous bazedoxifene acetate. The pharmaceutical compositions of the present invention include powders, granulates, aggregates and compacted compositions. The pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like. The dosages include dosages suitable for oral, buccal, rectal, parenteral (including subcutaneous, intramuscular, and intravenous), inhalant and ophthalmic administration.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Capsule dosages will contain the solid composition within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric coating.

The following examples are provided to enable one skilled in the art to practice the invention and are merely illustrative of the invention. The examples should not be read as limiting the scope of the invention as defined in the feature and advantages.

### EXAMPLES

### Example 1

4.70 gm of bazedoxifene base was added into a clean, dry round bottom flask. 25 ml ethanol was added to the flask and stirred to obtain a solution of bazedoxifene. 0.6 gm of acetic acid in 5 ml ethanol was added dropwise to the bazedoxifene solution and the reaction mass was stirred for about 5 hours at room temperature. The reaction mass was concentrated under vacuum at about 50° C. to about 55° C. to get 5.0 gm of amorphous bazedoxifene acetate.

### Example 2

0.75 gm of bazedoxifene acetate was charged in a clean, dry round bottom flask. 7.5 ml of isopropyl alcohol was added to this and temperature was raised to about 80° C. to get clear solution. The solution was lyophilized to get 0.75 gm of amorphous bazedoxifene acetate.

### Example 3

0.75 gm of bazedoxifene acetate was charged in a round bottom flask and 15 ml of isopropyl acetate was added. The temperature was raised to about 65° C. to about 70° C. and stirred for about 30 minutes. The undissolved particles were filtered over hyflo bed and the clear filtrate was lyophilized to get 0.5 gm of amorphous bazedoxifene acetate.

We claim:

1. Amorphous bazedoxifene acetate.

2. The compound of claim 1, having an X-ray Diffraction (XRD) spectrum, substantially in accordance with FIG. 1; and a Differential Scanning Calorimetric (DSC) thermogram, substantially in accordance with FIG. 2.

3. The compound of claim 1, having characteristic Infrared (IR) spectroscopy peaks, substantially in accordance with FIG. 3; and a Thermogravimetric Analysis (TGA) thermogram, substantially in accordance with FIG. 4.

4. The compound of claim 1, which is substantially pure amorphous form, having less than about 5% of crystalline forms of bazedoxifene acetate.

5. The compound of claim 4, having less than about 5%, of any of crystalline forms A and B of bazedoxifene acetate.

6. Amorphous particles of bazedoxifene having a specific surface area from about 1 m²/g to about 3 m²/g, as measured by Brunauer-Emmett-Teller (BET) method.

7. The compound of claim 6, wherein 90% of the particles have a particle size less than 260 μm.

8. The compound of claim 7, further having plate-like crystal shape, as observed by scanning electron microscope (SEM), which is substantially in accordance with FIG. 5.

9. A process for preparing the amorphous bazedoxifene acetate, of claim 1, comprising:
a) providing a solution of bazedoxifene acetate in a solvent or mixture of solvents or aqueous mixtures thereof; and
b) removing the solvents to obtain the substantially pure amorphous bazedoxifene acetate
wherein the solvent is selected from ethyl alcohol, isopropyl alcohol and isopropyl acetate.

10. The process of claim 9, wherein reaction temperature is from about 25° C. to about reflux temperature of the solvent or mixture of solvents used.

11. The process of claim 9, wherein the solvent is removed by spray drying.

12. The process of claim 9, wherein the solvent is removed by lyophilisation.

13. The process of claim 9, wherein the solvent is removed by rotary evaporation.
